(19) United States
(12) Patent Application Publication
(10) Pub. No.: US 2006/0089391 A1
(43) Pub. Date: Apr. 27, 2006

(54) Treatment of cancer in pediatric patients

(76) Inventors: Gary Gordon, Highland Park, IL (US); Anne E. Hagey, Lake Forest, IL (US); Kysa A. Meek, McHenry, IL (US); Saul H. Rosenberg, Grayslake, IL (US)

Correspondence Address: ROBERT DEBERARDINE;ABBOTT LABORATORIES, 100 ABBOTT PARK ROAD, DEPT. 377/AP6A, ABBOTT PARK, IL, 60064-6008, US

(21) Appl. No.: 11/139,326

(22) Filed: May 27, 2005

Related U.S. Application Data
(60) Provisional application No. 60/575,577, filed on May 28, 2004.

(51) Int. Cl. A61K 31/44

(52) U.S. Cl. 514/352

(57) ABSTRACT

Compositions comprising a cancer drug, a continuous oral dosing schedule with a drug which binds to the colchicine site of tubulin β-subunits, and methods of treating cancer in a pediatric patient using continuous dosing schedules are disclosed.

This application claims priority to U.S. Provisional Application 60/575,577, filed May 28, 2004, the specification of which is hereby incorporated by reference into this application.

### FIELD OF THE INVENTION

This invention pertains to a composition comprising a cancer drug, a continuous oral dosing schedule with the drug, and methods of treating cancer in pediatric patients using the drug.

### BACKGROUND OF THE INVENTION

The efficacy of many commercially-available cancer drugs for pediatric cancer patients is compromised by the necessity of intermittent administration due to severity of coincident adverse side effects. There is therefore an existing need in the therapeutic arts for improved treatment of cancer with drugs in pediatric patients.

### SUMMARY OF THE INVENTION

One embodiment of this invention, therefore, pertains to a continuous oral dosing schedule for treatment of cancer in a pediatric patient with a therapeutically acceptable amount of a drug, or a therapeutically acceptable salt thereof, which binds to tubulin β-subunits, wherein said dosing schedule lasts for at least five days.

Another embodiment pertains to a continuous oral dosing schedule for treatment of cancer in a pediatric patient with a therapeutically acceptable amount of a drug, or a therapeutically acceptable salt thereof, which binds to tubulin β-subunits, wherein said dosing schedule lasts for at least five days and during which the severity of at least one adverse side effect selected from the group consisting of constipation, elevated levels of serum transaminases, motor neuropathy, nausea, and sensory neuropathy, when compared to the severity of the same side effect coincident with treatment of the substantially same cancer with a parenterally administered drug which binds to tubulin p-subunits.

Still another embodiment pertains to a continuous oral dosing schedule for treatment of cancer in a pediatric patient with a therapeutically acceptable amount of a drug which binds to the colchicine site of tubulin β-subunits, or a therapeutically acceptable salt thereof, wherein said dosing schedule lasts for at least five days.

Still another embodiment pertains to a continuous oral dosing schedule for treating cancer in a pediatric patient with a therapeutically acceptable amount of a drug, or a therapeutically acceptable salt thereof, which binds to the colchicine site of tubulin β-subunits, wherein said dosing schedule lasts for at least five days and during which the severity of at least one adverse side effect selected from the group consisting of constipation, elevated levels of serum transaminases, motor neuropathy, nausea, and sensory neuropathy is essentially reduced when compared to the severity of the same side effect coincident with treatment of the substantially same cancer with a parenterally administered drug which binds to tubulin β-subunits.

Still another embodiment of this invention, therefore, pertains to a continuous oral dosing schedule for treatment of cancer in a pediatric patient with a therapeutically acceptable amount of N-(2-((4-hydroxyphenyl)amino)pyrid-3-yl)-4-methoxy-benzenesulfonamide, or a therapeutically acceptable salt thereof, wherein said dosing schedule lasts for at least five days.

Still another embodiment pertains to a continuous oral dosing schedule for treatment of cancer in a pediatric patient with a therapeutically acceptable amount of N-(2-((4-hydroxyphenyl)-amino)pyrid-3-yl)-4-methoxybenzenesulfonamide, or a therapeutically acceptable salt thereof, wherein said dosing schedule lasts for at least five days, and during which the severity of at least one adverse side effect selected from the group consisting of constipation, elevated levels of serum transaminases, motor neuropathy, nausea, and sensory neuropathy is essentially reduced when compared to the severity of the same side effect coincident with treatment of the substantially same cancer with a parenterally administered drug which binds to tubulin β-subunits.

Still another embodiment pertains to a method for treatment of cancer in a pediatric patient, said method comprising continuously orally administering, for at least five days, a therapeutically acceptable amount of a drug, or a therapeutically acceptable salt thereof, which binds to tubulin β-subunits.

Still another embodiment pertains to a method for treatment of cancer in a pediatric patient, said method comprising continuously orally administering, for at least five days, a therapeutically acceptable amount of a drug, or a therapeutically acceptable salt thereof, which binds to tubulin β-subunits, during which dosing schedule the severity of at least one adverse side effect selected from the group consisting of constipation, elevated levels of serum transaminases, motor neuropathy, nausea, and sensory neuropathy is essentially reduced when compared with treatment of the substantially same cancer with a parenterally administered drug which binds to tubulin β-subunits.

Still another embodiment pertains to a method for treatment of cancer in a pediatric patient, said method comprising continuously orally administering, for a time period of at least five days, a therapeutically acceptable amount of a drug, or a therapeutically acceptable salt thereof, which binds to the colchicine site of tubulin β-subunits.

Still another embodiment pertains to a method for treatment of cancer in a pediatric patient, said method comprising continuously orally administering, for at least five days, a therapeutically acceptable amount of a drug, or a therapeutically acceptable salt thereof, which binds to the colchicine site of tubulin β-subunits, during which dosing schedule, the severity of at least one adverse side effect selected from the group consisting of constipation, elevated levels of serum transaminases, motor neuropathy, nausea, and sensory neuropathy is essentially reduced when compared to the severity of the same side effect coincident with treatment of the substantially same cancer with a parenterally administered drug which binds to tubulin β-subunits.

Still another embodiment pertains to a method for treatment of cancer in a pediatric patient, said method comprising continuously orally administering, for at least five days, a therapeutically acceptable amount of N-(2-((4-hydroxyphenyl)-amino)pyrid-3-yl)-4-methoxybenzenesulfonamide, or a therapeutically acceptable salt thereof.

Still another embodiment pertains to a method for treatment of cancer in a pediatric patient, said method comprising continuously orally administering, for at least five days, a therapeutically acceptable amount of N-(2-((4-hydroxyphenyl)-amino)pyrid-3-yl)-4-methoxybenzenesulfonamide, or a therapeutically acceptable salt thereof, during which dosing schedule, the severity of at least one adverse side effect selected from the group consisting of constipation, elevated levels of serum transaminases, motor neuropathy, nausea, and sensory neuropathy is essentially reduced when compared to the severity of the same side effect coincident with treatment of the substantially same cancer with a parenterally administered drug which binds to tubulin β-subunits.

Still another embodiment pertains to a composition for immediate gastrointestinal release of N-(2-((4-hydroxyphenyl)amino)pyrid-3-yl)-4-methoxybenzenesulfonamide comprising a therapeutically effective amount of N-(2-((4-hydroxyphenyl)amino)pyrid-3-yl)-4-methoxybenzenesulfonamide and an excipient, which composition induces, upon continuous oral ingestion, essentially reduced severity of at least one side effect selected from the group consisting of constipation, elevated levels of serum transaminases, motor neuropathy, nausea, and sensory neuropathy and neutropenia when compared to the severity of the same side effect coincident with treatment of the substantially same cancer with a parenterally administered tubulin β-subunit binder.

Still another embodiment pertains to a composition for immediate release of N-(2-((4-hydroxyphenyl)amino)pyrid-3-yl)-4-methoxybenzenesulfonamide in the gastrointestinal environment for treatment of cancer in a pediatric patient, said composition comprising a therapeutically effective amount of N-(2-((4-hydroxyphenyl)amino)pyrid-3-yl)-4-methoxybenzenesulfonamide and an excipient which induces, upon continuous oral ingestion of one 75 mg per mm² per day dose for 21 days, a maximum plasma concentration of 8.0±1.7 μg/mL at 2.7±0.6 minutes and an area under the plasma concentration time curve of 52±21 μg·h/mL.

Still another embodiment pertains to a composition for immediate release of N-(2-((4-hydroxyphenyl)amino)pyrid-3-yl)-4-methoxybenzenesulfonamide in the gastrointestinal environment for treatment of cancer in a pediatric patient, said composition comprising a therapeutically effective amount of N-(2-((4-hydroxyphenyl)amino)pyrid-3-yl)-4-methoxybenzenesulfonamide and an excipient which induces, upon continuous oral ingestion of one 100 mg per mm² per day dose for 21 days, a maximum plasma concentration of 10.6±3.0 μg/mL at 1.9±1.0 minutes and an area under the plasma concentration time curve of 60±19 μg·h/mL.

Still another embodiment pertains to a composition for immediate release of N-(2-((4-hydroxyphenyl)amino)pyrid-3-yl)-4-methoxybenzenesulfonamide in the gastrointestinal environment for treatment of cancer in a pediatric patient, said composition comprising a therapeutically effective amount of N-(2-((4-hydroxyphenyl)amino)pyrid-3-yl)-4-methoxybenzenesulfonamide and an excipient which induces, upon continuous oral ingestion of one 130 mg per mm² per day dose for 21 days, a maximum plasma concentration of 10.8±2.9 μg/mL at 2.2±0.8 minutes and an area under the plasma concentration time curve of 62±19 μg·h/mL.

Still another embodiment pertains to a composition for immediate release of N-(2-((4-hydroxyphenyl)amino)pyrid-3-yl)-4-methoxybenzenesulfonamide in the gastrointestinal environment for treatment of cancer in a pediatric patient, said composition comprising a therapeutically effective amount of N-(2-((4-hydroxyphenyl)amino)pyrid-3-yl)-4-methoxybenzenesulfonamide and an excipient which induces, upon continuous oral ingestion of one 165 mg per mm² per day dose for 21 days, a maximum plasma concentration of 13.5±4.6 μg/mL at 3.0±1.7 minutes and an area under the plasma concentration time curve of 90±27 μg·h/mL.

Still another embodiment pertains to a composition for immediate release of N-(2-((4-hydroxyphenyl)amino)pyrid-3-yl)-4-methoxybenzenesulfonamide in the gastrointestinal environment for treatment of cancer in a pediatric patient, said composition comprising a therapeutically effective amount of N-(2-((4-hydroxyphenyl)amino)pyrid-3-yl)-4-methoxybenzenesulfonamide and an excipient which induces, upon continuous oral ingestion of one 200 mg per mm² per day dose for 21 days, a maximum plasma concentration of 16.9±8.1 μg/mL at 3.0±1.7 minutes and an area under the plasma concentration time curve of 91±24 μg·h/mL.

Still another embodiment pertains to a composition for immediate release of N-(2-((4-hydroxyphenyl)amino)pyrid-3-yl)-4-methoxybenzenesulfonamide in the gastrointestinal environment for treatment of cancer in a pediatric patient, said composition comprising a therapeutically effective amount of N-(2-((4-hydroxyphenyl)amino)pyrid-3-yl)-4-methoxybenzenesulfonamide and an excipient which induces, upon continuous oral ingestion of one 250 mg per mm² per day dose for 21 days, a maximum plasma concentration of 23.3±6.5 μg/mL at 2.0 minutes and an area under the plasma concentration time curve of 129±23 μg·h/mL.

Still another embodiment pertains to a continuous oral dosing schedule for treatment of cancer in a pediatric patient with 200 mg/mm²/day of N-(2-((4-hydroxyphenyl)amino)pyrid-3-yl)-4-methoxybenzenesulfonamide, wherein said dosing schedule lasts for the first 7 days of a 21 day schedule.

Still another embodiment pertains to a pharmaceutical composition having therapeutic synergy comprising N-(2-((4-hydroxyphenyl)amino)pyrid-3-yl)-4-methoxybenzenesulfonamide and at lease one cancer drug selected from the group consisting of cisplatin, docetaxel, and 5-fluorouracil.

Still another embodiment pertains to a method of treating cancer in a pediatric patient comprising administering a therapeutically effective amount of N-(2-((4-hydroxyphenyl)-amino)pyrid-3-yl)-4-methoxybenzenesulfonamide and at lease one additional drug selected from the group consisting of cisplatin, docetaxel, and 5-fluorouracil.

### DETAILED DESCRIPTION OF THE INVENTION

The term “at least five days,” as used herein, means the time period over which the drug is administered. In a preferred embodiment for the practice of this invention, at least five days means for the first 7 days of a 21 day schedule, for the first 14 days of a 21 day schedule, for he first 15 days of a 21 day schedule, for the first 21 days of a 28 day schedule, for 5 days then cessation for 5 days then continuation for 5 days then cessation for 5 days, i.e. (5 days on/5 days off)×2, and for 7 days then cessation for 7 days then continuation for 7 days then cessation for 7 days, i.e. (7 days on/7 days off)×2.

The term “coichicine site binder,” as used herein, means a tubulin β-subunit binder which binds to the colchicine site of the tubulin β-subunits and thereby inhibits the polymerization of tubulin.

A preferred example of a drug which binds to the colchicine site of tubulin β-subunits for the practice of this invention is N-(2-((4-hydroxyphenyl)amino)pyrid-3-yl)-4-methoxybenzenesulfonamide, also referred to herein as ABT-751. The synthesis of N-(2-((4-hydroxyphenyl)amino)pyrid-3-yl)-4-methoxybenzenesulfonamide is taught in U.S. Pat. No. 5,292,758, column 23, line 61 to column 24, line 12, hereby incorporated by reference into this specification.

The term “cancer,” as used herein, means brain tumor, Ewing's sarcoma, neuroblastoma, osteosarcoma, Rhabdomyosarcoma, and cancers resulting from the metastasis of disease from these areas.

The term “continuous,” as used herein, means at least once per day without missing a day.

The term “drug,” as used herein, means a compound which is suitable for prevention or treatment of disease or inhibition of one or more adverse physiological events.

The term “DLT,” as used herein, means dose-limiting toxicity.

The term “pediatric patient,” as used herein, means a human of about 5 to 18 years of age.

The term “essentially reduced,” as used herein in reference to severity of an adverse side effect means at least about 50% of the patient population tested did not experience that side effect at the Grade III or IV level, preferably about 75% of the patient population tested did not experience that side effect at the Grade III or IV level, more preferably about 85% of the patient population tested did not experience that side effect at the Grade III or IV level, even more preferably, about 95% of the patient population tested did not experience that side effect at the Grade III or IV level, and most preferably, 100% of the patient population tested did not experience that side effect at the Grade III or IV level.

Binding affinities of N-(2-((4-hydroxyphenyl)amino)pyrid-3-yl)-4-methoxybenzenesulfonamide, vinblastine, and paclitaxel were evaluated using the competition of [³H]colchicine to biotinylated bovine brain tubulin in a scintillation proximity assay.

TABLE 1
Inhibition Constants of ABT-751 and Other Tubulin β-Subunit
Binders in Binding Experiments with Bovine Brain Tubulin

| Compound | Kᵢ (μM) |
|---|---|
| ABT-751 | 2.60 (n = 4) |
| colchicine | 0.78 (n = 7) |
| paclitaxel | >100 (n = 4) |
| vinblastine | >100 (n = 4) |

The data in TABLE 1 demonstrate that N-(2-((4-hydroxyphenyl)amino)pyrid-3-yl)-4-methoxybenzenesulfonamide displaces [³H]colchicine from the coichicine site of tubulin β-subunits and is therefore a colchicine site binder.

The data in TABLE 1 also demonstrate that vinblastine and paclitaxel do not displace [³H]colchicine, are therefore not colchicine-site binders, and therefore must bind to tubulin β-subunits sites which are different from the colchicine binding site.

N-(2-((4-hydroxyphenyl)amino)pyrid-3-yl)-4-methoxybenzenesulfonamide is a colchicine site binder and exemplifies drugs which are useful for treatment of diseases which may be treated with coichicine-site binders other than colchicine itself.

The effectiveness of coichicine-site binders as drugs which are useful for treatment of diseases in humans depends on variables such as the composition comprising the drug, its route of administration, the amount of drug administered, and the dosing schedule. This invention pertains to an unexpected and surprising combination of variables which lead to a favorable therapeutic event with a sufficient reduction in the severity of at least one adverse side effect selected from the group consisting of anemia, alopecia, fluid retention, myelosupression, neuropathy and neutropenia as compared to the same side effect coincident with treatment of the substantially same disease with a parenterally administered drug which binds to tubulin β-subunits.

The tubulin β-subunit binders of this invention can be administered with or without an excipient. Excipients include encapsulating materials or additives such as absorption accelerators, antioxidants, binders, buffers, coating agents, coloring agents, diluents, disintegrants, emulsifiers, extenders, fillers, flavoring agents, humectants, lubricants, perfumes, preservatives, propellants, releasing agents, sterilizing agents, sweeteners, solubilizers, wetting agents and mixtures thereof. Excipients for solid dosage forms the tubulin β-subunit binders of this invention to be administered orally include agar, alginic acid, aluminum hydroxide, benzyl alcohol, benzyl benzoate, 1,3-butylene glycol, castor oil, cellulose, cellulose acetate, cocoa butter, corn starch, corn oil, cottonseed oil, ethanol, ethyl cellulose, ethyl laureate, ethyl oleate, gelatin, germ oil, glucose, glycerol, groundnut oil, isopropanol, isotonic saline, lactose, magnesium hydroxide, magnesium stearate, malt, olive oil, peanut oil, potassium phosphate salts, potato starch, propylene glycol, Ringer's solution, talc, tragacanth, water, safflower oil, sesame oil, sodium carboxymethyl cellulose, sodium lauryl sulfate, sodium phosphate salts, soybean oil, sucrose, tetrahydrofurfuryl alcohol and mixtures thereof. Excipients for the tubulin β-subunit binders of this invention to be administered ophthalmically or orally in liquid dosage forms include 1,3-butylene glycol, castor oil, corn oil, cottonseed oil, ethanol, fatty acid esters of sorbitan, germ oil, groundnut oil, glycerol, isopropanol, olive oil, polyethylene glycols, propylene glycol, sesame oil, water and mixtures thereof. Excipients for the tubulin β-subunit binders of this invention to be administered osmotically include chlorofluorohydrocarbons, ethanol, water and mixtures thereof. Excipients for the tubulin β-subunit binders of this invention to be administered parenterally include 1,3-butanediol, castor oil, corn oil, cottonseed oil, germ oil, groundnut oil, liposomes, oleic acid, olive oil, peanut oil, Ringer's solution, safflower oil, sesame oil, soybean oil, U.S.P. or isotonic sodium chloride solution, water and mixtures thereof. Excipients for the tubulin β-subunit binders of this invention to be administered rectally or vaginally include cocoa butter, polyethylene glycol, wax and mixtures thereof.

A preferable excipient for the practice of this invention using N-(2-((4-hydroxyphenyl)amino)pyrid-3-yl)-4-methoxybenzenesulfonamide is shown hereinbelow.

Formulation of N-(2-((4-hydroxyphenyl)amino)pyrid-3-yl)-4-
methoxybenzenesulfonamide

| Ingredient | % w/w | Purpose |
|---|---|---|
| ABT-751 | 30.0 | |
| microcrystalline cellulose | 15.8 | Filler |
| NF (Avicel ® PH101) | | |
| lactose monohydrate | 28.0 | Filler |
| povidone (USP, K29-32) | 8.0 | Binder |
| croscarmellose Na | 18.0 | Disintegrant |
| water | sufficient quantity | Binder Liquid |
| magnesium stearate | 0.2 | Lubricant |

A mixture of microcrystalline cellulose, N-(2-((4-hydroxyphenyl)amino)pyrid-3-yl)-4-methoxybenzenesulfonamide, lactose, and croscarmellose were granulated with a solution of povidone in water, dried, and milled. The milled product was blended with magnesium stearate.

The doses herein were made by filling capsules with the appropriate amount of blended product.

In accordance with routes of administration, the tubulin β-subunit binders of this invention may be administered orally, ophthalmically, osmotically, parenterally (subcutaneously, intramuscularly, intrastemally, intravenously), rectally, topically, transdermally, or vaginally. Orally administered solid dosage forms can be administered as capsules, dragees, granules, pills, powders, or tablets. Ophthalmically and orally administered dosage forms may be administered as elixirs, emulsions, microemulsions, suspensions, or syrups. Osmotically and topically administered dosage forms may be administered as creams, gels, inhalants, lotions, ointments, pastes, or powders. Parenterally administered dosage forms may be administered as aqueous or oleaginous suspensions. Rectally and vaginally dosage forms may be administered as creams, gels, lotions, ointments, or pastes.

For the practice of this invention, it is meant to be understood that while administration of drugs which bind to other than the colchicine binding site of tubulin β-subunits are preferentially administered parenterally, oral administration of drugs which bind to the colchicine binding site of tubulin β-subunits is more preferable than parenteral administration of the same drug.

The therapeutically acceptable amounts of the tubulin β-subunit binders of this invention and their dosing schedules depend on the recipient of treatment, the disease being treated and the severity thereof, the composition containing the tubulin β-subunit binder, the time of administration, the route of administration, the potency of the tubulin β-subunit binder, the rate of clearance of the tubulin β-subunit binder, and whether or not another drug is co-administered.

The daily amount of N-(2-((4-hydroxyphenyl)amino)pyrid-3-yl)-4-methoxybenzenesulfonamide to be administered orally in a continuous, once daily dose to pediatric patients for a continuous 7 day per 21 day dosing schedule was about 100 mg/mm², about 130 mg/mm², about 165 mg/mm², about 200 mg/mm², or about 250 mg/mm² with maximum daily doses of 150 mg/day, 200 mg/day, 250 mg/day, 300 mg/day, and 370 mg/day, respectively.

The daily amount of N-(2-((4-hydroxyphenyl)amino)pyrid-3-yl)-4-methoxybenzenesulfonamide to be administered orally in a continuous, once daily dose to pediatric patients for a continuous 21 day per 28 day dosing schedule was about 75 mg/mm², about 100 mg/mm², about 130 mg/mm², about 165 mg/mm², or about 200 mg/mm² with maximum daily doses of 125 mg/day, 150 mg/day, 200 mg/day, 250 mg/day, and 300 mg/day, respectively.

The dose escalation scheme is shown in TABLE 1.

TABLE 1

| | Daily × 7 Schedule | | Daily × 21 Schedule | |
|---|---|---|---|---|
| | | Eq. Adult | | Eq. Adult |
| | Daily Dose | Dose | Daily Dose | Dose |
| Dose Level | (mg/m²/d) | (mg/d) | (mg/m²/d) | (mg/d) |
| 1 | 100 | 180 | 75 | 130 |
| 2 | 130 | 234 | 100 | 180 |
| 3 | 165 | 297 | 130 | 234 |
| 4 | 200 | 360 | 165 | 360 |
| 5 | 250 | 450 | — | — |

Protocol-specific definitions of peripheral neuropathy grades were developed for children. ABT-751-related grade three or grade four non-hematologic toxicity is as defined in National Cancer Institute Common Toxicity Criteria version 2.0.

During the Daily ×7 Schedule DLT:

For the 10 mg/mm²/d dose level, no. DLT's per no. evaluations was 0/3.

For the 130 mg/mm²/d dose level, no. DLT's per no. evaluations was 1/6, wherein the DLT was persistent motor neuropathy (gr. 2).

For the 165 mg/mm²/d dose level, no. DLT's per no. evaluations was 1/6, wherein the DLT was persistconstipation (gr. 3).

For the 200 mg/mm²/d dose level, no. DLT's per no. evaluations was 1/8, wherein the DLT was pain (gr. 3) and cardiac (decreased shortening fraction).

For the 250 mg/mm²/d dose level, no. DLT's per no. evaluations was 2/3, wherein the DLT was fatigue (gr. 3, n=2) and cardiac (decreased shortening fraction), pain (gr. 3, n=1), cardiac (decreased shortening fraction, gr. 2, n=1), neutrophenia (gr. 3, n=1).

During the Daily ×21 Schedule DLT:

For the 75 mg/mm²/d dose level, no. DLT's per no. evaluations was 0/3.

For the 100 mg/mm²/d dose level, platelets which did not recover by day 42 (gr. 2).

For the 130 mg/mm²/d dose level, no. DLT's per no. evaluations was 3/6, fatigue, thrombocytopenia, constipation, nausea/vomiting, pain (each occurred in a single patient, all gr. 3).

For the 165 mg/mm²/d dose level, no. DLT's per no. evaluations was 3/3 fatigue, neuropathy, pain, nausea/vomiting/dehydration, neutropenia, and increased ALT (gr 3, n=1).

These results show the decreased occurrence of adverse side-effects in patients taking N-(2-((4-hydroxyphenyl)amino)pyrid-3-yl)-4-methoxybenzenesulfonamide orally.

Pharmacokinetic profiles of representative patients following dosing on with N-(2-((4-hydroxyphenyl)amino)pyrid-3-yl)-4-methoxybenzenesulfonamide are summarized in TABLE 2 (mean±SD).

TABLE 2

| Dose | | Cₘₐₓ | Tₘₐₓ | AUC |
|---|---|---|---|---|
| mg/mm²/d | N | (μg/mL) | (h) | (mcg/h/mL) |
| 75 mg QD | 3 | 8.0 ± 1.7 | 2.7 ± 0.6 | 52 ± 21 |
| 100 mg QD | 8 | 10.6 ± 3.0 | 1.9 ± 1.0 | 60 ± 19 |
| 130 mg QD | 6 | 10.8 ± 2.9 | 2.2 ± 0.8 | 62 ± 19 |
| 165 mg QD | 9 | 13.5 ± 4.6 | 3.0 ± 1.7 | 90 ± 27 |
| 200 mg QD | 6 | 16.9 ± 8.1 | 3.0 ± 1.7 | 91 ± 24 |
| 250 mg QD | 2 | 23.3 ± 6.5 | 2.0 | 129 ± 23 |

The preferred dose of N-(2-((4-hydroxyphenyl)amino)pyrid-3-yl)-4-methoxybenzenesulfonamide on the daily ×7 day schedule is 200 mg/mm²/d (substantially higher than the adult MTD of 130 200 mg/mm²/d) and that dose-limiting toxicities on the day 7 schedule include fatigue, neuropathy/neuropathic pain, nausea/vomiting/dehydration, constipation, neutropenia, and asymptomatic decrease in cardiac decreased shortening fraction.

It may also be concluded that the recommended dose of N-(2-((4-hydroxyphenyl)amino)pyrid-3-yl)-4-methoxybenzenesulfonamide on the daily ×21 day schedule is 100 mg/mm²/d (equivalent to the adult MTD of 110 mg/mm²/d) and that dose-limiting toxicities on the day 21 schedule include fatigue, neuropathy/neuropathic pain, nausea/vomiting/dehydration, elevated ALT, neutropenia and thrombocytopenia.

Patients (n=19) with neuroblastoma have experienced time-to-disease progresson with a median progression free survival (FPS) of 20 weeks.

Specifically, a five year old patient having neuroblastoma has continued on N-(2-((4-hydroxyphenyl)amino)pyrid-3-yl)-4-methoxybenzenesulfonamide with disease stabilization after 44 cycles of the 100 mg/mm²/d on the daily ×7 schedule with resolution of pain and subjective improvement in quality of life.

The foregoing is merely illustrative of the invention and is not intended to limit the invention to the disclosed embodiments. Variations and changes obvious to one skilled in the art are intended to be within the scope and nature of the invention which are defined in the appended claims.

What is claimed is:

1. A continuous oral dosing schedule for treatment of cancer in a pediatric patient with a therapeutically acceptable amount of a drug, or a therapeutically acceptable salt thereof, which binds to tubulin β-subunits, wherein said dosing schedule lasts for at least five days.

2. A continuous oral dosing schedule for treatment of cancer in a pediatric patient with a therapeutically acceptable amount of a drug, or a therapeutically acceptable salt thereof, which binds to tubulin β-subunits, wherein said dosing schedule lasts for at least five days and during which the severity of at least one adverse side effect selected from the group consisting of constipation, elevated levels of serum transaminases, motor neuropathy, nausea, and sensory neuropathy, when compared to the severity of the same side effect coincident with treatment of the substantially same cancer with a parenterally administered drug which binds to tubulin β-subunits.

3. A continuous oral dosing schedule for treatment of cancer in a pediatric patient with a therapeutically acceptable amount of N-(2-((4-hydroxyphenyl)amino)pyrid-3-yl)-4-methoxy-benzenesulfonamide, or a therapeutically acceptable salt thereof, wherein said dosing schedule lasts for at least five days.

4. A continuous oral dosing schedule for treatment of cancer in a pediatric patient with a therapeutically acceptable amount of N-(2-((4-hydroxyphenyl)-amino)pyrid-3-yl)-4-methoxybenzenesulfonamide, or a therapeutically acceptable salt thereof, wherein said dosing schedule lasts for at least five days, and during which the severity of at least one adverse side effect selected from the group consisting of constipation, elevated levels of serum transaminases, motor neuropathy, nausea, and sensory neuropathy is essentially reduced when compared to the severity of the same side effect coincident with treatment of the substantially same cancer with a parenterally administered drug which binds to tubulin β-subunits.

5. A composition for immediate release of N-(2-((4-hydroxyphenyl)amino)pyrid-3-yl)-4-methoxybenzenesulfonamide in the gastrointestinal environment for treatment of cancer in a pediatric patient, said composition comprising a therapeutically effective amount of N-(2-((4-hydroxyphenyl)amino)pyrid-3-yl)-4-methoxybenzenesulfonamide and an excipient which induces, upon continuous oral ingestion of one 130 mg per mm² per day dose for 21 days, a maximum plasma concentration of 10.8±2.9 μg/mL at 2.2±0.8 minutes and an area under the plasma concentration time curve of 62±19 μg·h/mL.

6. A continuous oral dosing schedule for treatment of cancer in a pediatric patient with 200 mg/mm² day of N-(2-((4-hydroxyphenyl)amino)pyrid-3-yl)-4-methoxybenzenesulfonamide, wherein said dosing schedule lasts for the first 7 days of a 21 day schedule.
